(19) United States
(12) Patent Application Publication
(10) Pub. No.: US 2024/0115540 A1
(43) Pub. Date: Apr. 11, 2024

(54) METHODS FOR PROVIDING HEALTH BENEFITS IN ANIMALS

(71) Applicant: Société des Produits Nestlé S.A., Vevey (CH)

(72) Inventors: Yuanlong Pan, Chesterfield, MO (US); Qinghong Li, Chesterfield, MO (US); Vincenzo Sorrentino, Lausanne (CH); Sonia Karaz, EPALINGES (CH)

(21) Appl. No.: 18/453,835

(22) Filed: Aug. 22, 2023

Related U.S. Application Data
(60) Provisional application No. 63/411,743, filed on Sep. 30, 2022.
(60) Provisional application No. 63/424,307, filed on Nov. 10, 2022.

(51) Int. Cl. A61K 31/23; A61K 31/197; A61K 31/198; A61K 31/202; A61K 31/355; A61K 31/375; A61K 31/4188; A61K 31/4415; A61K 31/455; A61K 31/519; A61K 31/525; A61K 31/714; A61P 13/12

(52) U.S. Cl. CPC A61K 31/23; A61K 31/197; A61K 31/198; A61K 31/202; A61K 31/355; A61K 31/375; A61K 31/4188; A61K 31/4415; A61K 31/455; A61K 31/519; A61K 31/525; A61K 31/714; A61P 13/12

(57) ABSTRACT

The present disclosure provides methods and compositions for treating glomerular hyperfiltration, reducing glycogen storage in tissues, or reducing uremic toxins in an animal, comprising orally administering a composition comprising medium chain triglycerides, eicosapentaenoic acid (EPA), docosahexaenoic acid (DHA), arginine, antioxidants, and B vitamins to the animal.

### CROSS REFERENCE TO RELATED APPLICATION

This application claims priority to U.S. Provisional Application Ser. No. 63/411,743 filed Sep. 30, 2022 and U.S. Provisional Application Ser. No. 63/424,307 filed Nov. 10, 2022, the disclosure of which is incorporated in its entirety herein by this reference.

### BACKGROUND

Renal failure is one of the most common causes of death in dogs and cats. In animals which suffer from renal disease, several blood indices are used to determine the severity of the disease. These indices include blood urea nitrogen (BUN) and creatinine. BUN and creatinine levels in the bloodstream increase during the course of renal failure because damage to the kidney of the animal makes the kidney inadequate to filter waste products. Because inadequate filtration of waste products is the fundamental basis of renal disease, BUN and creatinine are considered the primary indicators of renal disease.

Other clinical parameters which are important to the animal suffering from renal disease are phosphorus, carbon dioxide, and triglyceride levels. Hyperphosphatemia (abnormally high blood levels of phosphorus) often manifests itself during renal disease. Previous scientific research has indicated that lowering dietary intake of phosphorus is beneficial to lessening the progression of renal disease. However, prior commercial pet food formulations have been unable to provide low levels of dietary phosphorus and still meet all of the amino acid requirements of the canine and feline because the phosphorus component of such diets is primarily derived from ingredients high in protein. Thus, lowering the phosphorus content of the diet required a lowering of the protein components in the diet to levels which are insufficient to supply the amino acid requirements of the animal.

Carbon dioxide levels are an indicator of the level of metabolic buffering (acid-base balance) that occurs in an animal. Metabolic acidosis becomes a problem for an animal suffering from renal disease, and high carbon dioxide levels are indicators of a lack of buffering. Another parameter which is important in animals suffering from renal disease is the blood triglyceride level. It is important in the animal suffering from renal disease as the triglyceride level is often greater than in normal animals. It would be desirable to be able to control these additional parameters in the renal patient through diet.

Current dietary therapies to reduce BUN, creatinine, and phosphorus levels include decreasing the amount of dietary protein to levels where amino acids are present in insufficient quantities. Decreasing the level of dietary protein decreases BUN since urea is ultimately derived from protein. However, such diets may result in other problems developing for the animal as the animal's protein needs are unmet. Accordingly, there remains a need in this art for therapies which can provide treatment for kidney disease and associated symptoms.

### SUMMARY

The present disclosure relates to methods and compositions to provide a health benefit in an animal. More specifically, the present disclosure relates to compositions that comprise medium chain triglycerides (MCT), eicosapentaenoic acid (EPA), docosahexaenoic acid (DHA), antioxidants, arginine, and B vitamins. The present disclosure also relates to methods for treating glomerular hyperfiltration, reducing glycogen storage in tissues in an animal, or reducing uremic toxins in the animal, the method comprising orally administering a composition comprising MCT, EPA, DHA, arginine, antioxidants, and B vitamins to the animal.

Additional features and advantages are described herein and will be apparent from, the following Detailed Description.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight (wt %) of the total weight of the composition on a dry matter basis unless expressed otherwise. When reference is made to the pH, values correspond to pH measured at 25° C. with standard equipment.

The terms “food,” “food product” and “food composition” mean a product or composition that is intended for ingestion by an animal and provides at least one nutrient to the animal. The term “pet food” means any food composition intended to be consumed by a companion animal. Such food compositions can include main meal, treats, beverages, supplements, etc.

The term “companion animal” means a dog or a cat. As used herein, the term “cat” and “feline” can be used interchangeably. Additionally, the term “dog” and “canine” can be used interchangeably. In one embodiment, the companion animal can be a cat.

The term “recommended daily requirement” or “recommended daily allowance” or “RDA” refers to the daily amount of a vitamin or other nutrient recommended by a recognized agency or standard within the art including without limitation, National Institutes of Health (NIH), Institute of Medicine of the National Academies (TOM), Dietary Reference Intake (DRI) Nutrient Report, The European Pet Food Industry Federation (FEDIAF), or The Association of American Feed control Officials (AAFCO) as of Jan. 1, 2023. In one aspect, the RDA can refer to minimum levels defined for a cat and/or dog by AAFCO.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. Additionally, for example, if the average lifespan for a breed of cat is 15 years, then a cat within that breed greater than 7.5 years old would be considered “aging” for the purposes herein. In an embodiment, the compositions and methods disclosed herein involve a senior animal, e.g. a senior dog or a senior cat. Animals, such as dogs and cats, are considered senior in the last 25% of their lives. As noted herein, the life span of a dog or a cat depends on its size and/or its breed, and a senior dog or senior cat can be determined based on the above calculation but using the numerical value of 75% such that the age threshold is exceeding 75% of the average lifespan.

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%. “Kibbles” means pieces of dry or semi-moist pet food which can have a pellet shape or any other shape. Non-limiting examples of kibbles include particulates; pellets; pieces of pet food, dehydrated meat, meat analog, vegetables, and combinations thereof; and pet snacks, such as meat or vegetable jerky, rawhide, and biscuits.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Moreover, the description of some steps as “optional” does not imply that the other steps which are not explicitly described as optional are necessarily required.

Any embodiment disclosed herein can be combined with any other embodiment disclosed herein.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The relative terms “improved,” “increased,” “enhanced” and the like refer to the effects of the composition disclosed herein having a specific active or blend (e.g., a composition comprising a therapeutically effective amount of medium chain triglycerides or a prophylactic dose of medium chain triglycerides) relative to a composition having a lower amount or lacking the active or blend, (e.g., medium chain triglycerides), but otherwise identical.

The term “therapeutically effective amount” means an amount of a compound of the present invention that (i) treats or prevents the particular disease, condition, or disorder, (ii) attenuates, ameliorates, or eliminates one or more symptoms relating thereto of the particular disease, condition, or disorder, or (iii) prevents or delays the onset of one or more symptoms of the particular disease, condition, or disorder described herein. In one embodiment, therapeutically effect amount can refer to the amount of a composition, active, or blend to treat any condition relating to renal diseases in animals.

The term “long-term administration” means periods of repeated administration or consumption in excess of one month. Periods of longer than two, three, or four can be used for certain embodiments. Also, more extended periods that include longer than 5, 6, 7, 8, 9, or 10 months can be used. Periods in excess of 11 months or 1 year can also be used. Longer term use extending over 1, 2, 3, or more years can also be included in the invention. For certain aging animals, the animal will continue consuming on a regular basis for the remainder of its life. Sometimes this is referred to as consumption for “extended” periods.

The term “regular basis” means at least monthly dosing with the compositions or consumption of the compositions, and in some aspects, weekly dosing. More frequent dosing or consumption, such as twice, three, or seven times weekly, can be used in certain embodiments. Still other embodiments include regimens that comprise at least once daily consumption. The skilled artisan will appreciate that dosing frequency will be a function of the composition that is being consumed or administered, and some compositions may require more or less frequent administration to maintain a desired level of hydration.

A “medium chain triglyceride” is a lipid in which three medium-chain fatty acids are bound by ester linkages to a glycerol backbone, and at least two and preferably all three of the fatty acids are each between six and twelve carbons in length. The medium-chain fatty acids are caproic acid (comprising six carbon atoms or C6:0), caprylic acid (comprising eight carbon atoms or C8:0), capric acid (comprising ten carbon atoms or C10:0) and lauric acid (comprising twelve carbon atoms or C12:0). In one embodiment, the medium chain fatty acids of the medium chain triglycerides can include at least 50% caprylic acid of the total medium chain fatty acids that are present. In another embodiment, the medium chain fatty acids of the medium chain triglycerides can include at least 90% caprylic acid of the total medium chain fatty acids that are present.

### Embodiments

The present disclosure relates to methods and compositions that provide a health benefit in an animal. More specifically, in one embodiment, a composition can comprise medium chain triglycerides (MCT), eicosapentaenoic acid (EPA), docosahexaenoic acid (DHA), arginine, antioxidants, and B vitamins. In another embodiment, a method for treating glomerular hyperfiltration, reducing glycogen storage in tissues in an animal, or reducing uremic toxins in the animal can comprise orally administering a composition comprising MCT, EPA, DHA, arginine, antioxidants, and B vitamins to the animal. In one aspect, the composition can be administered in a therapeutically effective amount.

The present inventors have discovered that the present compositions can treat glomerular hyperfiltration and/or reduce glycogen storage and/or reduce uremic toxins in an animal in need thereof. Such effect can help treat such animals suffering from kidney disease, obesity, diabetes, metabolic syndrome, hyalinosis, hypertension, hypothyroidism, neuropathy, cardiovascular disease, and combinations thereof.

The methods generally comprise orally administering to the animal a composition comprising medium chain triglycerides, eicosapentaenoic acid (EPA), docosahexaenoic acid (DHA), arginine, antioxidants, and B vitamins. In one embodiment, the composition can further comprise other omega-3 fatty acids, antioxidants, amino acids, and mixtures thereof. In one aspect, the composition comprises a preservative. In various aspects, the composition can be a pet food, such as a wet pet food, a semi-moist pet food, or a dry pet food, e.g., kibble; or a supplement, or treat.

Generally, the medium chain triglycerides can be about 0.1 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, from about 1 wt % to about 7 wt %, or from about 2 wt % to about 10 wt % of the composition. The medium chain triglycerides may be prepared by any known process, such as direct esterification, rearrangement, fractionation and/or transesterification. For example, the medium chain triglycerides may be prepared from a source of vegetable oil, such as coconut oil, through a rearrangement process. The chain length and distribution thereof may vary depending on the source oil. For example, such MCTs can include those having at least 50%, at least 55%, at least 60%, at least 65%, at least 70%, at least 75%, at least 80%, at least 85%, at least 95%, or even 100%, caprylic acid.

Non-limiting examples of suitable omega-3 fatty acids include eicosapentaenoic acid (EPA), docosahexaenoic acid (DHA), alpha-linolenic acid (ALA), and mixtures thereof. In one embodiment, the omega-3 fatty acid(s) can range from about 0.1 wt % to about 5 wt % of the composition. In some embodiments, the omega-3 fatty acid(s) are at least about 0.1 wt %, at least about 0.5 wt %, at least about 1.0 wt %, or at least 2.0 wt %. In one embodiment, the DHA can be present in the composition in an amount from about 0.1% to about 5%. In other aspects, the DHA can be present from about 0.1 wt %, 0.5 wt %, 1 wt %, 1.5 wt %, 2 wt %, 2.5 wt %, or 3 wt % to about 0.5 wt %, 1 wt %, 1.5 wt %, 2 wt %, 2.5 wt %, 3 wt %, 3.5 wt %, 4 wt %, 4.5 wt %, or 5 wt %. In another embodiment, the EPA can be present in the composition in an amount from about 0.05 wt % to about 15 wt %. In other aspects, the EPA can be present from about 0.1 wt %, 0.5 wt %, 1 wt %, 1.5 wt %, 2 wt %, 2.5 wt %, or 3 wt % to about 0.5 wt %, 1 wt %, 1.5 wt %, 2 wt %, 2.5 wt %, 3 wt %, 3.5 wt %, 4 wt %, 4.5 wt %, or 5 wt %.

Non-limiting examples of suitable B vitamins include B1 (thiamin), B2 (riboflavin), B3 (niacin) B5 (pantothenic acid or pantothenate) B6 (pyridoxine) B7 (biotin) B9 (folate or folic acid) B12 (cobalamin). In one embodiment, the B vitamins can be present from about 1 to 100 times of the recommended daily requirements (RDA). In various aspects, the B vitamins can be present from about 1, 5, or 10 to about 15, 30, 60, or 80 times of the RDA to about 20, 40, 80 times of the RDA. In one embodiment, the niacin can be present from about 1 to about 30 times of RDA, the pantothenate can be present from about 1 to about 50 times of the RDA, the pyridoxine can be present from about 1 to about 30 times of the RDA, the thiamine can be present from about 1 to about 200 times of the RDA, the riboflavin can be present from about 1 to about 100 times of the RDA, the biotin can be present from about 1 to about 40 times of the RDA, the folic acid can be present from about 1 to about 50 times of the RDA, and the cobalamin can be present from about 1 to about 50 times of the RDA. In other aspects, the niacin can be present from about 1, 2, 3, 4, 5, 10, 15, or 20 to about 2, 3, 4, 5, 10, 15, 20, 25 or 30 times of the RDA; the pantothenate can be present from about 1, 2, 3, 4, 5, 10, or 15 to about 2, 3, 4, 5, 10, 15, 20, 30, or 50 times of the RDA; the pyridoxine can be present from about 1, 2, 3, 4, 5, 10, 15, or 20 to about 2, 3, 4, 5, 10, 15, 20, 25, or 30 times of the RDA; the thiamine can be present from about 1, 2, 3, 4, 5, 10, 15, 20, or 40 to about 2, 3, 4, 5, 10, 15, 20, 60, 100, or 200 times of the RDA; the riboflavin can be present from about 1, 2, 3, 4, 5, 10, 15, 20, or 30 to about 2, 3, 4, 5, 10, 15, 20, 25, 50, 80, or 100 times of the RDA; the biotin can be present from about 1, 2, 3, 4, 5, 10, 15, or 20 to about 2, 3, 4, 5, 10, 15, 20, 25, or 40 times of the RDA; the folic acid can be present from about 1, 2, 3, 4, 5, 10, 15, or 20 to about 2, 3, 4, 5, 10, 15, 20, 25, 30, or 50 times of the RDA; and/or the cobalamin can be present from about 1, 2, 3, 4, 5, 10, 15, or 20 to about 2, 3, 4, 5, 10, 15, 20, 25, 30, or 50 times of the RDA. The RDA levels can be converted from dosage to specific amounts of the composition by adding the RDA per kilogram of the composition.

Generally, the composition can also comprise antioxidants including but not limited to vitamin C, vitamin E, carotenoids such as beta-carotene and lycopene, and phenolic compounds such as quercetin, catechins, resveratrol, coumaric acid, anthocyanins.

In one embodiment, the compositions can include arginine, vitamin C, and vitamin E. Generally, arginine includes any forms of arginine, and/or any substrates of nitric oxide synthase, and/or any substrates that can increase arginine level in the body. In one embodiment, the arginine can be about 1 wt % to about 10 wt % of the composition. In other aspects, the arginine can be present from about 1 wt %, 2 wt %, or 3 wt % to about 5 wt %, 8 wt %, or 10 wt % of the composition. In one embodiment, the vitamin E can be about 1 to about 50 times of the RDA. In other aspects, the vitamin E can be present about 1, 2, 3, 4, 5, or 10 to about 2, 3, 4, 5, 10, 15, 20, 25, 30, or 50 times of the RDA of the composition. In one embodiment, the vitamin C can be about 0.001 wt % to about 1 wt % of the composition. In other aspects, the vitamin C can be present about 0.001 wt %, 0.005 wt %, or 0.01 wt % to about 0.5 wt %, 0.8 wt %, or 1 wt % of the composition.

In some embodiments, the composition can be administered to the animal for a time period of at least one week, at least one month, at least two, three, four, five or six months; and in some embodiments, for at least one year. During the time period, the composition can be administered to the animal at least one day per week, at least two days per week, at least three, four, five or six days per week; or even seven days per week. The composition can be administered in a single dose per day or in multiple separate doses per day. In one embodiment, MCT can be about 0.1 wt % to about 50 wt % of the composition. In other aspects, MCT can be present about 0.1 wt %, 1 wt %, or 3 wt % to about 5 wt %, 10 wt %, 15 wt %, or 20 wt % of the composition. In another embodiment, the composition can be administered in an amount that provides about 10 mg to 5 g of MCTs per kg body weight of the animal per day. In one aspect, 10 mg to about 500 mg of MCTs per kg body weight of the animal can be administered per day.

In one specific embodiment, the animal can be a companion animal. In one aspect, the companion animal can be a cat. In another aspect, the companion animal can be a dog. In one embodiment, the animal can be a senior animal or an aging animal. In one aspect, the animal can be a senior cat or senior dog. In another aspect, the animal can be an aging cat or aging dog.

In one embodiment, the compositions described herein can be pet food compositions. The pet food compositions disclosed herein can be any food formulated for consumption by a pet such as a cat. In an embodiment, the pet food composition provides complete nutrition as defined by the Association of American Feed Control Officials (AAFCO) as of Jan. 1, 2023, and which depends on the type of animal for which the composition is intended (e.g., a cat). In another embodiment, the composition can be a supplement. Such a supplement can be added to a food composition or be administered in conjunction with a food composition, or administered separately. As such, in some embodiments, the present compositions can be complete and nutritionally balanced pet foods.

Generally, pet food composition can comprise protein, carbohydrates, fat, and ash. In various embodiments, the pet food compositions comprise from about 15% to about 50% crude protein. In some embodiments, such compositions may further comprise from about 5% to about 40% fat. In other embodiments, the compositions may further comprise from about 15% to about 60% carbohydrate. In other embodiments, the composition may further comprise from about 0.1% to about 15% ash.

The pet food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

The pet food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as starches, humectants, oral care ingredients, preservatives, other amino acids, other antioxidants, fibers, prebiotics, sugars, animal oils, aromas, other oils additionally or alternatively to vegetable oil, salts, vitamins, minerals, probiotic microorganisms, bioactive molecules or combinations thereof.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

Yet another aspect of the present disclosure is a method of making a pet food, the method comprising adding medium chain triglycerides, eicosapentaenoic acid (EPA), docosahexaenoic acid (DHA), arginine, vitamin E, vitamin C, and B vitamins to at least one other comestible ingredient, where the medium chain triglycerides, eicosapentaenoic acid (EPA), docosahexaenoic acid (DHA), arginine, vitamin E, vitamin C, and B vitamins are added in an amount effective to provide the health benefits as disclosed herein. For example, the medium chain triglycerides, eicosapentaenoic acid (EPA), docosahexaenoic acid (DHA), arginine, vitamin E, vitamin C, and B vitamins can be added in a therapeutically effect amount such that administration of the pet food is effective to treat or prevent glomerular hyperfiltration, reducing glycogen storage, or reduce uremic toxins in an animal.

### EXAMPLE

By way of example and not limitation, the following non-limiting study is illustrative of compositions and methods using MCTs for treating glomerular hyperfiltration, reducing glycogen storage in tissues, or reduce uremic toxins in an animal, in one or more embodiments provided by the present disclosure.

### Example 1—CKD Mice Study

The present study used db/db mice at 8 weeks of age for 8 weeks of intervention. The mice were divided into 4 groups of 9-10 mice administered with different blends (the Control group was administered with standard AIN-93M diet, the Dapa Group was administered standard AIN-93M augmented with dapagliflozin at 10 mg/kg, the RPB group was administered a standard diet augmented with a renal protection blend, the RPB+ group was administered a standard diet augmented with a renal protection blend that included medium chain triglycerides); the diets are shown in Table 1. Additionally, a group of 9 db/db control mice was administered a standard AIN-93M diet; also shown in Table 1. The blends were administered to study glomerular hyperfiltration and glycogen storage in tissues. Final weekly food intake, final body weight, final blood glucose level, final lean body mass, and final fat mass of the groups is presented in Table 2. Also presented in table 2 were GFR progression, urine albumin/creatine ratio, urine protein/creatinine ratio, glomerulosclerosis score, Periodic acid-Schiff (PAS) staining scores for distal convoluted tubule (DCT) lumen, PAS staining scores for intranuclear inclusions distal tubules, and PAS staining scores for cytoplamis aggregates distal tubules.

TABLE 1

| Ingredient | Control | Dapa | RPB | RPB+ | db/m | |
|---|---|---|---|---|---|---|
| (g/kg diet) | Group | Group | Group | Group | Group | |
| Casein | 140 | 140 | 118 | 118 | 140 | Control |
| Corn Starch | 495.69 | 495.69 | 494.69 | 469.69 | 495.69 | diet |
| Maltodextrin 10 | 125 | 125 | 125 | 125 | 125 | AIN-93M |
| Sucrose | 100 | 100 | 100 | 100 | 100 | |
| Cellulose, BW200 | 50 | 50 | 50 | 50 | 50 | |
| Mineral mix | 35 | 35 | 35 | 35 | 35 | |
| Vitamin mix | 10 | 10 | 10 | 10 | 10 | |
| L-Cystine | 1.8 | 1.8 | 1.8 | 1.8 | 1.8 | |
| Choline bitartrate | 2.5 | 2.5 | 2.5 | 2.5 | 2.5 | |
| Soybean oil | 40 | 40 | 20 | 20 | 40 | |
| tBHQ | 0.008 | 0.008 | 0.008 | 0.008 | 0.008 | |
| Menhaden Oil, | 0 | 0 | 20 | 20 | 0 | Added to |
| 200 ppm | | | | | | RPB/RPB+ |
| BHT | | | | | | groups |
| L-Arginine- | 0 | 0 | 20 | 20 | 0 | |
| Vitamin E acetate | 0 | 0 | 2.2 | 2.2 | 0 | |
| (500 IU/g 50% ) | | | | | | |
| Ascorbic acid | 0 | 0 | 0.47 | 0.47 | 0 | |
| phosphate | | | | | | |
| (33% Vitamin C) | | | | | | |
| Niacin | 0 | 0 | 0.045 | 0.045 | 0 | |
| Ca Pantothenate | 0 | 0 | 0.034 | 0.034 | 0 | |
| Pyridoxine HCl | 0 | 0 | 0.023 | 0.023 | 0 | |
| Thiamine HCl | 0 | 0 | 0.014 | 0.014 | 0 | |
| Riboflavin | 0 | 0 | 0.009 | 0.009 | 0 | |
| Folic Acid | 0 | 0 | 0.003 | 0.003 | 0 | |
| Biotin (1%) | 0 | 0 | 0.081 | 0.081 | 0 | |
| Vitamin B12, 0.1% | 0 | 0 | 0.2 | 0.2 | 0 | |
| MCT (100%) | 0 | 0 | 0 | 25 | 0 | |

TABLE 2

| | Control | Dapa | RPB | RPB+ | db/m |
|---|---|---|---|---|---|
| Ingredients | Group | Group | Group | Group | Group |
| Food intake (g) | 6.21 | 6.71 | 6.51 | 6.54 | 3.1 |
| Body weight (g) | 34.82 | 40.94 | 34.66 | 34.65 | 34.85 |
| Blood glucose | 599 | 464.78 | 618.5 | 571.20 | 147.75 |
| (mg/dL) | | | | | |
| Lean mass (g) | 13.77 | 15.31 | 13.80 | 14.47 | 19.33 |
| Fat mass (g) | 19.47 | 23.99 | 18.31 | 17.53 | 12.48 |
| GFR progression | 0.06 | 0.01 | 0.03 | 0.02 | 0.00 |
| (ml/min) | | | | | |
| Albumin/Creatinine | 295.03 | 181.54 | 233.46 | 214.78 | 20.15 |
| ratio | | | | | |
| Protein/Creatinine | 61.72 | 36.92 | 46.42 | 46.02 | 3.13 |
| ratio | | | | | |
| Glomerulosclerosis | 1.91 | 1.83 | 1.72 | 1.47 | 0.29 |
| score | | | | | |
| PAS DCT lumen | 1.56 | 1.44 | 0.8 | 0.7 | 0.00 |
| PAS intranuclear | 2.44 | 2.11 | 2.4 | 1.8 | 0.5 |
| inclusions DT | | | | | |
| PAS cytoplasmic | 2.33 | 1.78 | 2.1 | 1.9 | 0.75 |
| aggregates DT | | | | | |

In addition to the measurements of Table 2, the mice were measured for hyalinosis on a scoring system of no hyalinosis, intermediate hyalinosis, or severe hyalinosis, where hyalinosis refers to the thickening of the walls of the glomerular arterioles by the deposits of homogenous hyaline material. The RPB+ group was the only group with mice not having severe hyalinosis other than the db/m mice. The control group had 1 mouse with no hyalinosis, 4 mice with intermediate hyalinosis, and 4 with significant hyalinosis; the dapa group had 2 mice with no hyalinosis, 5 mice with intermediate hyalinosis, and 2 mice with significant hyalinosis, the RPB group had 5 mice with no hyalinosis, 4 mice with intermediate hyalinosis, and 1 mouse with significant hyalinosis, and the RPB+ group had 2 mice with no hyalinosis and 8 mice with intermediate hyalinosis.

The impact of RPB and RPB+ on food intake was evaluated over the treatment period. As shown in table 2, the final food intake of db/db mice treated with RPB and RPB+ was similar to db/db mice. Consequently, there was no significant difference in food intake between RPB and RPB+ treated db/db mice and Vehicle-treated db/db mice. The impact of RPB and RPB+ on body weight was evaluated over the treatment period. As shown in table 2, the final body weight of RPB and RPB+ treated db/db mice did not significantly differ from those of Vehicle-treated db/db mice. RPB and RPB+ showed no significant difference in blood glucose levels with Vehicle-treated db/db. RPB+ treated db/db mice showed an increase in lean mass from Vehicle-treated db/db mice. RPB and RPB+ treated db/db mice showed normalization of GFR progression. RPB and RPB+ treated db/db mice showed improvement in urinary albumin/creatinine ratio. RPB and RPB+ treated db/db mice showed improvement in urinary protein/creatinine ratio. RPB+ treated db/db mice showed a significant improvement of glomerulosclerosis in kidney tissues compare to Vehicle-treated db/db mice. RPB treated db/db mice showed improvement in DCT lumen, and RPB+ treated db/db mice showed a significant improvement of DCT lumen in kidney tissues compare to Vehicle-treated db/db mice. RPB+ treated db/db mice showed improvement in PAS intranuclear inclusions distal tubules in kidney tissues compare to Vehicle-treated db/db mice. RPB+ treated db/db mice showed a significant improvement on PAS cytoplasmic aggregates distal tubules in kidney tissues compare to Vehicle-treated db/db mice.

Additionally, uremic toxins were measured for the db/db mice for the Control group (vehicle), the RPB group and the RPB+ group. Results are shown as medians for each group in Table 3.

TABLE 3

| | Median Concentration (ug/mL) | | |
|---|---|---|---|
| Uremic toxin | Control | RPB | RPB+ |
| trimethylamine N-oxide | 23.04 | 5.15 | 4.75 |
| p-cresol sulfate | 0.22 | 0.22 | 0.19 |
| indoxyl sulfate | 35.82 | 29.93 | 27.16 |

As shown in Table 3, the RPB and RPB+ compositions reduced levels of uremic toxin with the RPB+ being the most effective. As discussed herein, the difference between the RPB and RPB+ diets was solely due to the inclusion of MCTs in the RPB+. As such, the present data shows that MCTs are particularly effective in reducing uremic toxins, which was surprising and unexpected.

### Example 2—CKD Cat Study

Serum and urine samples were collected from 28 healthy cats, 5 cats with stage 1 CKD, and 11 cats with stage 2 CKD. Cats with CKD were diagnosed and staged according to the IRIS guidelines (http://www.iris-kidney.com/guidelines/staging.html). The samples were stored in −80° C. until use. Targeted analysis was performed to determine the concentrations of four uremic toxins at Colorado State University. The authentic standards p-cresol sulfate (pCS) was purchased from APExBio Technology, indoxyl sulfate (IS) and IS-D5 from Cayman Chemical, trimethylamine N-oxide (TMAO) from Sigma-Aldrich, TMAO-D9 from Santa Cruz Biotechnology, pCS-D7 from Cambridge Isotope, phenyl sulfate (PS) from TCI Chemicals. Uremic toxin internal standard mix contained 12.5 μg/mL of TMAO-D9, 250 μg/mL of pCS-D7, 12 μg/mL of IS-D5, and 1 μg/mL of IAA (indole 3-acetic acid)-D5 dissolved in 50% methanol in water.

The serum sample (40 μL) was mixed with 10 μL of the internal standard and 200 μL of cold methanol. The mixture was vortexed for 5 seconds and then incubated at −20° C. overnight, followed by centrifugation at 15000 g and 4° C. for 15 min. The supernatant (100 μL) was recovered and then added with 900 μL of 50% methanol, which were then stored at −20° C. until analysis. A small aliquot of sample extract was pooled from each sample to generate a quality control (QC) sample.

The urine sample (20 μL) was mixed with 80 μL of cold methanol, and then incubated at −20° C. overnight, followed by centrifugation at 15000 g and 4° C. for 15 min. The supernatant (10 μL) was recovered and then added with 10 μL of internal standard and 1 mL of 50% methanol, which were then stored at −20° C. until analysis. A small aliquot of sample extract was pooled from each sample to generate a quality control (QC) sample.

UPLC-MS/MS was performed on a Waters Acquity UPLC coupled to a Waters Xevo TQ-S triple quadrupole mass spectrometer. Chromatographic separations were carried out on a Waters UPLC T3 stationary phase (2.1×50 mm, 1.8 μM) column. All Raw data files were imported into the Skyline open-source software package. Each target analyte was visually inspected for retention time and peak area integration. Peak areas were extracted for target compounds detected in biological samples and normalized to the peak area of the appropriate internal standard or surrogate in each sample. Absolute quantitation (ug/mL) was calculated using the linear regression equation generated for each compound from the calibration curve.

Results of the samples are provided in Tables 4 and 5. Table 4 shows serum uremic toxin levels for 28 healthy control cats (CON) vs 5 cats with stage 1 CKD (CKD1) vs 11 cats with stage 2 CKD (CKD2). Table 5 shows urine uremic toxic levels of 27 healthy control cats (CON), 5 cats with stage 1 CKD (CKD1), 11 cats with stage 2 CKD (CKD2) normalized by urine creatinine concentration of each cat; data shown as means.

TABLE 4

| | Mean Concentration (ug/mL) | | |
|---|---|---|---|
| Uremic toxin | CON | CKD1 | CKD2 |
| trimethylamine N-oxide | 0.66 | 1.23 | 3.27 |
| phenyl sulfate | 0.3 | 1.91 | 2.98 |
| p-cresol sulfate | 3.36 | 3.03 | 13.63 |
| indoxyl sulfate | 1.19 | 2.07 | 5.37 |

TABLE 5

| | Mean Concentration (ug/mL) | | |
|---|---|---|---|
| Uremic Toxin | CON | CKD1 | CKD2 |
| trimethylamine N-oxide | 0.44 | 1.21 | 1.24 |
| phenyl sulfate | 0.16 | 0.5 | 0.42 |
| p-cresol sulfate | 0.78 | 1.92 | 2.26 |
| indoxyl sulfate | 0.66 | 2.7 | 1.47 |

As shown in tables 4 and 5, uremic toxins were increased in circulation and in urine in proportion to the severity of CKD in cats. Excess uremic toxins are known to lead to chronic inflammation, excess free radical production, insulin resistance, cellular apoptosis, and compromised intestinal barrier, which can lead to further renal disease. As such, cats having CKD would benefit from the RPB and RPB+ compositions disclosed herein.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for treating glomerular hyperfiltration, reducing glycogen storage in tissues, or reduce uremic toxins in an animal, the method comprising orally administering a composition comprising medium chain triglycerides, eicosapentaenoic acid (EPA), docosahexaenoic acid (DHA), arginine, antioxidants, and B vitamins to the animal.

2. The method of claim 1, wherein the B vitamins include niacin, pantothenate, pyridoxine, thiamine, riboflavin, biotin, folic acid, and cobalamin.

3. The method of claim 2, wherein the niacin is about 1 to about 30 times of RDA, the pantothenate is about 1 to about 50 times of the RDA, the pyridoxine is about 1 to about 30 times of the RDA, the thiamine is about 1 to about 200 times of the RDA, the riboflavin is about 1 to about 100 times of the RDA, the biotin is about 1 to about 40 times of the RDA, the folic acid is about 1 to about 50 times of the RDA, and the cobalamin is about 1 to about 50 times of the RDA.

4. The method of claim 1, wherein the medium chain triglycerides are about 0.1 wt % to about 60 wt % of the composition.

5. The method of claim 1, wherein the medium chain triglycerides are about 1 wt % to about 20 wt % of the composition

6. The method of claim 1, wherein the medium chain triglycerides include a medium-chain fatty acid selected from the group consisting of caprylic acid, capric acid, and a mixture thereof.

7. The method of claim 1, wherein the DHA is about 0.1 wt % to about 5.0 wt % of the composition, the EPA is about 0.1 wt % to about 5.0 wt % of the composition, the arginine is about 1 wt % to about 10 wt % of the composition, the vitamin E is about 1 to about 50 times of the RDA, the vitamin C is about 0.001 wt % to 1 wt % of the composition, and the B vitamins are about 1 to 100 times of the RDA.

8. The method of claim 1, wherein the composition further comprises an additional omega-3 fatty acid, an antioxidant, a preservative, or mixtures thereof.

9. The method of claim 1, wherein the composition is a complete and nutritionally balanced pet food.

10. The method of claim 1, wherein the composition is a supplement.

11. The method of claim 1, wherein the animal is a companion animal.

12. The method of claim 1, wherein the animal has a health condition selected from the group consisting of: kidney disease, obesity, diabetes, metabolic syndrome, hyalinosis, hypertension, hypothyroidism, neuropathy, cardiovascular disease, and combinations thereof.

13. The method of claim 1, wherein the animal is a senior animal.

14. The method of claim 1, wherein the composition is administered to the animal daily for at least one week.

15. The method of claim 1, wherein the composition is administered in an amount that provides about 10 mg to about 500 mg of the MCTs per kg body weight of the animal per day.
